(19) United States
(12) Patent Application Publication
(10) Pub. No.: US 2021/0115521 A1
(43) Pub. Date: Apr. 22, 2021

(54) METHOD FOR INHIBITING TUMOR PROGRESSION OR DETERMINING TUMOR PROGRESSION STATE IN GASTRIC CANCER

(71) Applicant: National Tsing Hua University, Hsinchu City (TW)

(72) Inventors: Wen-Ching Wang, Taichung City (TW); Lin-Lu Tseng, Hsinchu County (TW); Ta-Sen Yeh, Taipei City (TW); Hsin-Hung Cheng, Tainan City (TW); Chiou-Hwa Yuh, Hsinchu City (TW)

(73) Assignee: National Tsing Hua University, Hsinchu City, TW (TW)

(21) Appl. No.: 17/074,647

(22) Filed: Oct. 20, 2020

Related U.S. Application Data
(60) Provisional application No. 62/924,678, filed on Oct. 22, 2019.

(51) Int. Cl. C12Q 1/6886; A61K 31/496; A61K 31/553; C12Q 1/686; A61P 35/04

(52) U.S. Cl. CPC C12Q 1/6886; A61K 31/496; A61K 31/553; C12Q 2600/158; A61P 35/04; C12Q 2600/118; C12Q 1/686

(57) ABSTRACT

Provided is a method of inhibiting tumor progression in a subject suffering from gastric cancer, including administering to said subject a pharmaceutical composition including an inhibitor of targeting PHF8-c-Jun-PKCα-Src-PTEN axis, or a pharmaceutically acceptable salt thereof. A method of determining a tumor progression state in a subject suffering from gastric cancer is also provided, which comprises providing a sample from the subject; detecting PHF8 expression level in the sample from the subject; and determining the tumor progression state of gastric cancer by the PHF8 expression level, wherein the PHF8 expression level is positively detected from moderate to strong expression indicating the subject suffering a late stage of gastric cancer.

### CROSS-REFERENCES TO RELATED APPLICATIONS

The present application claims priority to U.S. Provisional Appl. No. 62/924,678, filed Oct. 22, 2019, and contains a Sequence Listing in a computer readable form, which are incorporated herein by reference in their entirety.

### FIELD OF THE INVENTION

The present invention relates to a method of inhibiting or determining tumor progression in a subject suffering from gastric cancer.

### DESCRIPTION OF PRIOR ART

Gastric cancer (GC) is the second leading cause of death among all malignancies worldwide. More than 50% of new GC cases occur in the WHO Western Pacific region. The staging system for gastric cancer is often followed by the American Joint Committee on Cancer (AJCC) TNM system, which includes three key feathers: the extent (size) of the tumor (T); the spread to nearby lymph nodes (N); and the spread (metastasis) to distant sites (M). According to the TNM system, there are five stages: stage 0 (zero) and stages I through IV (1 through 4). The stage provides a common way of describing the cancer.

Surgical resection remains the gold standard in GC therapy, particularly for early-stage GC. However, GC is generally asymptomatic at the early stage; as such, more than 40% of patients with GC are diagnosed with metastatic disease at the first visit. The prognosis of metastatic GC remains poor, with a median survival of 4.3 months for patients who receive best supportive care and 11 months for those who receive combination chemotherapy. The survival of patients with GC treated with chemotherapy for the last two decades has remained steady owing to a death of major breakthroughs in the development of new cytotoxic agents. A recent trend is to combine targeted therapy with chemotherapy. In a multi-center ToGA study, the median overall survival was 13.8 months in an anti-HER2 targeted treatment (trastuzumab) group as compared with 11.1 months in a chemotherapy alone group, suggesting that patients with HER2-positive GC (12-20%) may benefit from this approach. However, no effective targeted treatments are available for advanced HER2-negative cases.

Genome-based molecular characterization provides a new avenue for patient stratification, prognosis, and the customization of treatment. The Cancer Genome Atlas data for 295 primary GC tissues without chemotherapy and radio-therapy reveal differential patterns of DNA methylation, somatic gene alterations, gene expression, and proteomic events. Key genetic alternations are primarily found in oncogenes/tumor suppressor genes, including TP53, KRAS, ARID1A, PIK3CA, ERBB3, PTEN, and HLA-B. A global gene-expression profiling analysis and targeted sequencing of 300 GC samples by the Asian Cancer Research Group corroborated common recurrent driver mutations, including mutations in TP53, ARID1A, PIK3CA, KRAS, PTEN and ERBB3.

Apart from mutational patterns revealed from these frontier studies, epigenetic irregularities that contribute to progression and even chromosome remodeling and increased instability cannot be overlooked. Among epigenetic regulators, histone lysine demethylases (KDMs) have drawn substantial attention, as they catalyze the removal of key methyl groups from histones, which can greatly impact gene expression and the chromatin spatial organization and even rewire tumorigenic programs with increased malignant capability.

Plant homeodomain (PHD) finger protein 8 (PHF8, also termed KDM7B) is a member of the histone demethylase family. Several studies have showed that PHF8 is overexpressed in several malignancies, including prostate cancer, esophagus cancer, lung cancer, laryngeal and hypopharyngeal squamous cell carcinoma, acute lymphoblastic leukemia, and GC suggesting that PHF8 serves as a potential oncogenic epigenetic regulator. However, the underlying mechanism of PHF8 is involved in regulating GC progression is still needed to investigate.

### SUMMARY OF THE INVENTION

The present invention provides a method of inhibiting tumor progression in a subject suffering from gastric cancer, comprising administering to said subject a pharmaceutical composition comprising an inhibitor of targeting PHF8-c-Jun-PKCα-Src-PTEN axis, or a pharmaceutically acceptable salt thereof.

The present invention also provides a method determining a tumor progression state in a subject suffering from gastric cancer, comprising: (a) providing a sample from the subject; (b) detecting PHF8 expression level in the sample from the subject; and (c) determining the tumor progression state of gastric cancer by the PHF8 expression level, wherein the PHF8 expression level is positively detected from moderate to strong expression indicating the subject suffering a late stage of gastric cancer.

### BRIEF DESCRIPTION OF THE DRAWINGS

FIG. 1. The results of analysis of KDMs associated with worse clinical outcomes in HER2-negative GC. FIG. 1A shows Kaplan-Meier curves for 5-year overall survival and FIG. 1B shows Kaplan-Meier curves for first progression in HER2-negative GC patients stratified by the expression level of KDM1B. Patients were divided into high-expression (High, solid line) and low-expression (Low, dot line) groups based on the best performing threshold used as a cutoff (FDR=0.1). Statistical significance was determined by log-rank test.

FIG. 2. Elevated expression of PHF8 was associated with GC metastasis. FIG. 2A shows the expression of PHF8 in gastric mucosa (N), primary site tumor (P) and metastatic site tumor (M) from Oncomine database. **p<0.01 (two-tailed Student's t test). FIG. 2B shows the comparison of PHF8 expression between tumor (T) and adjacent normal (N) tissues from the selected study using Oncomine. **p<0.01 (paired t test).

FIG. 3. PHF8 was crucial for MKN28 cell proliferation and migration in vitro and in vivo. FIG. 3A. shows the analysis of PHF8 in MKN28 infected with lentivirus carrying control pLKO or shPHF8 constructs (#1 or #2) by Western blotting. Actin was the internal control. FIG. 3B shows the depletion of PHF8 exhibited a reduced degree of cell proliferation in MKN28. FIG. 3C shows images of control and shPHF8 MKN28-luc xenografts after 6 wk of injection. FIG. 3D shows the tumor volumes were measured every week up to 6 wk. Data are presented as mean±SD, with the SD derived from five mice. **P<0.01 (one-way ANOVA test). FIG. 3E shows the depletion of PHF8 exhibited a reduced degree of cell migration in MKN28. The depletion of PHF8 exhibited a significantly reduced proportion of embryos with migration behavior in MKN28 in zebrafish migration assays. FIG. 3F shows representative fluorescence images of a zebrafish embryo displaying cell dissemination (Upper) or no migration (Lower) at 3 dpi (Scale bar, 100 μm) and FIG. 3G shows the quantified results. Total number of embryos is shown in the bracket. *P<0.05 (two-tailed Student's t test). Data from FIGS. 3B-3G are presented as the average of three replicates ±SD*P<0.05, **P<0.01 (two-tailed Student's t test).

FIG. 4. PHF8 was crucial for MKN45 cell proliferation and migration in vitro and in vivo. FIG. 4A shows the analysis of PHF8 abundance in MKN45 infected with lentivirus carrying control pLKO or shPHF8 constructs (#1 or #2), followed by puromycin selection. Lysates were analyzed by western blotting. FIG. 4B shows the depletion of PHF8 exhibited a reduced degree of cell proliferation in MKN45. FIG. 4C shows the depletion of PHF8 exhibited a reduced degree of cell migration in MKN45 using trans-well cell migration assay. Fluorescence microscopic analysis was conducted at 1 dpi and 3 dpi. FIG. 4D shows the representative fluorescence images of a zebrafish embryo displaying cell dissemination (upper panel) or no migration (lower panel) at 3 dpi. Scale bar, 100 μm. FIG. 4E shows the depletion of PHF8 exhibited a reduced proportion of embryos with migration behavior in MKN45. Total number of embryos is shown in the bracket. Data were obtained from 3 independent studies. *p<0.05, **p<0.01 (two-tailed Student's t test). Data from FIGS. 4B-C are presented as the average of 3 replicates ±SD. **p<0.01 (two-tailed Student's t test).

FIG. 5. PHF8 regulated the expression of genes involved in cell migration and cell motility. FIG. 5A shows DAVID functional annotation analysis of the genes with twofold or less alterations in PHF8-knockdown MKN28 in a microarray analysis. FIG. 5B shows qRT-PCR analysis of down-regulated genes in the cell migration/cell motility/cell motion category. The data were normalized by GAPDH mRNA levels. FIG. 5C shows ChIP analysis of PHF8 occupancy on the promoter region of genes involved in cell migration category in MKN28 using IgG or anti-PHF8 antibodies. Data are presented as the average of three replicates ±SD*P<0.05, **P<0.01 (two-tailed Student's t test).

FIG. 6. The results of analysis of PHF8 occupancy in the promoter region of PRKCA. FIG. 6A shows ChIP analysis of PHF8 enrichment in the promoter region of PRKCA with four sets of primers as indicated. IgG as a control. FIGS. 6B-D show fold enrichment of PHF8, H3K9me2, and H4K20me1 on the PRKCA promoter in pLKO and shPHF8 (#1 and #2) MKN28 across three regions. (FIG. 6B: −1,100 to −906, FIG. 6C: −932 to −797, and FIG. 6D: −925 to −744). Data from FIGS. 6A-6D are presented as the average of three replicates ±SD*P<0.05, **P<0.01 (two-tailed Student's t test).

FIG. 7. PKCα acted as a downstream target of PHF8/c-JUN to promote GC progression. FIG. 7A shows analysis of ectopic expression of PKCα in pLKO, shPHF8 #1, or shPHF8 #2 MKN28. Cells were transfected with control (ctl) or a PKCα (PRKCA) expression vector, followed by western blotting analysis. FIG. 7B shows the ectopic expression of PKCα significantly restored the degree of cell proliferation in shPHF8 #1 and shPHF8 #2, respectively. FIG. 7C shows the ectopic expression of PKCα in shPHF8 MKN28 significantly rescued the degree of migratory activity. Data from FIGS. 7B-7C are presented as the average of 3 replicates ±SD. **p<0.01 (two-tailed Student's t test).

FIG. 8. PKCα acted as a downstream target of PHF8/c-JUN to promote GC progression in MKN45. FIG. 8A shows the ectopic expression of PKCα in pLKO, shPHF8 #1, or shPHF8 #2 MKN45. Cells were transfected with control (ctl) or a PKCα (PRKCA) expression vector, followed by western blotting analysis. FIG. 8B shows the ectopic expression of PKCα significantly restored the degree of cell proliferation in shPHF8 #1 and shPHF8 #2, respectively. FIG. 8C shows the ectopic expression of PKCα in shPHF8 MKN45 significantly rescued the degree of migratory activity. FIG. 8D shows endogenous association between PHF8 and c-Jun. MKN45 cell lysates were used for IP assays with rabbit-IgG, anti-PHF8, or anti-c-Jun. FIG. 8E shows ChIP analysis of c-Jun enrichment on the PRKCA promoter in PHF8-depleted cells. FIG. 8F shows the AP-1 reporter activity of MKN45 cells (pLKO, shPHF8 #1, or shPHF8 #2) co-transfected with an AP-1 reporter plasmid and an internal β-galactosidase control plasmid. Data form FIGS. 8B-8C and 8E-8F are presented as the average of 3 replicates ±SD. **p<0.01 (two-tailed Student's t test).

FIG. 9. ICAM-1 was a downstream target of PHF8 to promote migratory activity. FIG. 9A shows qRT-PCR analysis of ICAM-1 in pLKO, shPHF8 #1, or shPHF8 #2 MKN45. The data were normalized by GAPDH mRNA levels. FIG. 9B shows ChIP analysis of PHF8 enrichment on the ICAM-1 promoter in pLKO and shPHF8 (#1 and #2) MKN28 and MKN45, respectively. FIG. 9C shows the depletion of ICAM-1 exhibited a reduced degree of migratory activity in MKN28 and MKN45, respectively.

FIG. 10. PHF8 interacted with c-JUN and regulates AP-1 reporter activity. FIG. 10A shows endogenous association between PHF8 and c-Jun. MKN28 cell lysates were used for IP assays with IgG, anti-PHF8, or anti-c-Jun. FIG. 10B shows ChIP analysis of c-Jun enrichment on the PRKCA promoter in PHF8-depleted cells. FIG. 10C shows the AP-1 reporter activity of cells (pLKO, shPHF8 #1, or shPHF8 #2) cotransfected with an AP-1 reporter plasmid and an internal β-galactosidase control plasmid. FIG. 10D shows the AP-1 reporter activity of MKN28 cotransfected with vectors [Flag, wild-type PHF8(WT), or inactive PHF8(H247A), and HA, or HA-c-JUN, plus a β-galactosidase internal control] as indicated. Data from FIGS. 10B-10D are presented as the average of three replicates ±SD*P<0.05, **P<0.01 (two-tailed Student's t test).

FIG. 11. The results of mapping the interaction region between PHF8 and c-Jun. Co-IP assays were performed with anti-Flag and anti-HA in HEK293T cells co-transfected with each of Flag-PHF8 and HA-c-Jun plasmids as indicated. (FIG. 11A: IP with anti-Flag antibody; FIG. 11B: IP with anti-HA antibody)

FIG. 12. The results of Micro-western-array analysis. The heatmap of the fold changes of signaling proteins between pLKO, shPHF8 #1 and shPRKCA #2 MKN28.

FIG. 13. The PHF8-PKCα axis regulated PTEN destabilization by SRC activation. FIG. 13A shows PTEN protein level in shPHF8 (#1 and #2) and shPRKCA (#1 and #2) MKN28 with or without MG132 treatment. FIG. 13B shows abundance of Src and pSrc (Y419) in pLKO or shPHF8 (#1 or #2) transfected with a control (ctl) or a PRKCA-expressing vector. FIG. 13C shows PTEN protein level in shPRKCA (#1 and #2) cells transfected with a kinase-active Src (Y419D) or a kinase dead Src (Y419F) vector.

FIG. 14. PHF8-PKCα axis regulated PTEN destabilization by SRC activation. FIG. 14A shows PTEN protein level in shPHF8 (#1 and #2) and shPRKCA (#1 and #2) MKN28 with or without MG132 treatment. FIG. 14B shows abundance of Src and pSrc (Y419) in pLKO or shPHF8 (#1 or #2) MKN28 transfected with a control (ctl) or a PRKCA-expressing vector. FIG. 14C. shows PTEN protein level in shPRKCA (#1 and #2) MKN28 cells transfected with a kinase-active Src (Y419D) or a kinase-dead Src (Y419F) vector.

FIG. 15. Pharmacological inhibition of PHF8-PKCα-Src-PTEN blocks GC progression in vitro and in vivo. FIG. 15A shows PTEN protein level in MKN28 treated with 0, 1, 2, or 4 μM of Midostaurin (Mido). FIG. 15B shows migration activity of MKN28 treated with Mido by using Transwell migration assay. FIG. 15C shows PTEN protein level in MKN28 treated with 0, 1, 2, or 4 μM of Bosutinib (Bosu). FIG. 15D shows migration activity of MKN28 treated with Bosu. Data from FIGS. 15B and 15D are presented as the average of three replicates ±SD **P<0.01 (two-tailed Student's t test). Zebrafish xeno-transplantation assays using Mido or Bosu. FIG. 15E shows the representative fluorescence images of a zebrafish embryo displaying cell dissemination (Upper) or no migration (Lower) at 3 dpi. Cyan, blood vessel. (Scale bar, 100 μm.) FIG. 15F shows the quantification of embryos with migration behavior in vehicle or drug-treated groups. Total number of embryos used in vehicle, Mido, and Bosu is shown in the bracket. Data were obtained from three independent studies. **P<0.01 (two-tailed Student's t test). FIG. 15G shows a schematic diagram of the PHF8-c-Jun complex in contribution of GC progression.

FIG. 16. Pharmacological inhibition of PHF8-PKCα-Src-PTEN blocks GC progression in vitro and in vivo. FIG. 16A shows analysis of PTEN protein level in MKN45 treated with 0, 1, 2 or 4 μM of Midostaurin (Mido). FIG. 16B shows migration activity of MKN45 treated with Mido by using trans-well migration assay. FIG. 16C shows PTEN protein level in MKN45 treated with 0, 1, 2 or 4 μM of Bosutinib (Bosu). FIG. 16D shows migration activity of MKN45 treated with Bosu by using trans-well migration assay. Data form FIGS. 16B and 16D are presented as the average of 3 replicates ±SD. *p<0.05, **p<0.01 (two-tailed Student's t test). Zebrafish xenotransplantation assays using Mido or Bosu. Fluorescence microscopic analysis was conducted at 1 dpi and 3 dpi. FIG. 16E shows the representative fluorescence images of a zebrafish embryo displaying cell dissemination (upper panel) or no migration (lower panel) at 3 dpi. Cyan, blood vessel. Scale bar, 100 μm. FIG. 16F shows the quantification of embryos with migration behavior in vehicle or drug-treated groups. Total number of embryos used in vehicle, Mido, Bosu is shown in the bracket. Data were obtained from 3 independent studies. *p<0.05 (two-tailed Student's t test).

FIG. 17. Pharmacological inhibition of PHF8-PKCα-Src-PTEN blocked tumor growth of MKN28 xenografts in vivo. MKN28 cells were implanted subcutaneously in nude mice. When tumors were staged to 100±30 mm³, mice were treated with vehicle, Midostaurin (50 mg/kg) and Bosutinib (50 mg/kg), respectively. Images of MKN28-luc xenografts were taken after seven weeks of implantation and the tumor volumes were measured every one week up to seven weeks. Data are presented as mean±SD, with the SD derived from five mice. **p<0.01 (One-way ANOVA test).

FIG. 18. The sublethal dosage of Midostaurin combined with Bosutinib in zebrafish embryos. The Midostaurin at 187.5 nM combined with Bosutinib at 1.25 μM had no toxicity for zebrafish embryos treated for 2 days starting 3 dpf. Therefore, we choosed this dosage for xenotransplanation assay.

FIG. 19. Midostaurin had synergetic effect with Bosutinib for MKN28 to inhibit the cell proliferation and migration in vivo. FIG. 19A shows MKN28 proliferation assays in zebrafish xenotransplantation model. Percentage of change in area were calculated by the formula (3 dpi−1 dpi)/1 dpi. FIG. 19B shows percentage of change of intensity were calculated by the formula (3 dpi−1 dpi)/1 dpi. FIG. 19C shows the representative fluorescence images of zebrafish embryo at 1 dpi versus 3 dpi for DMSO, Mido, Bosu, or Mido+Bosu were shown. FIG. 19D shows the percentage of embryos with migration behavior. Fluorescence microscopic analysis was conducted at 1 dpi and 3 dpi. FIG. 19E shows the representative fluorescence images of a zebrafish embryo displaying cell dissemination (upper panel) or no migration (lower panel) at 3 dpi.

FIG. 20. The clinical relevance of PHF8, PKCα, and PTEN in 42 GC subjects obtained from CGMH. FIG. 20A shows the representative images of immunohistochemical profiles. PHF8, PKCα, and PTEN were immunostained for each of gastric tissue specimens (n=42). (Scale bar, 100 μm.) FIG. 20B shows the IRS score of GC samples. The correlation of IHC signals for two-group comparisons. FIG. 20C shows the correlation of PHF8 and PKCα, FIG. 20D shows the correlation of PHF8 and PTEN, and FIG. 20E shows the correlation of PKCα and PTEN (E). Statistical significance was evaluated using the x 2 test. FIG. 20F shows PHF8 expression is significantly correlated with tumor stage in patients with GC. Statistical calculation is conducted using one-way ANOVA analysis. FIG. 20G shows five-year OS and 5-y DFS analysis according to the level of PHF8 and PKCα expression in patients with GC (n=42). High, IRS≥8; low, IRS≤6. Statistical significance (PHF8^{high}PKCα^{high} vs. PHF8^{low}PKCα^{low}) was determined by log-rank test.

FIG. 21. The clinical relevance of PHF8, PKCα, and PTEN in 42 GC subjects purchased from Biomax tissue array ST1505. FIG. 21A shows the representative image of Immunohistochemical profiles. PHF8, PKCα and PTEN were immunostained for each of gastric tissue specimens (n=50). Scale bar, 100 μm. The correlation of IHC signals between two groups. FIG. 21B shows the correlation of PHF8 and PKCα (B), FIG. 21C shows the correlation of PHF8 and PTEN (C), and FIG. 21D shows the correlation of PKCα and PTEN. Statistical significance was evaluated using the Chi-square test. FIG. 21E shows PHF8 expression is significantly correlated with tumor Stage in patients with GC. Statistical calculation is conducted using one-way ANNOVA analysis.

### DETAILED DESCRIPTION OF THE INVENTION

In the present invention, PHD finger protein 8 (PHF8, or KDM7B) is significantly associated with poor survival in HER2-negative GC. The depletion of PHF8 significantly reduced cancer progression in GC cells and in mouse xenografts. PHF8 regulates genes involved in cell migration/motility based on a microarray analysis. Furthermore, PHF8 interacts with c-Jun on the promoter of PRKCA encoding PKCα. The depletion of PHF8 or PKCα greatly upregulated PTEN expression and reduced Src activation, which can be rescued by ectopic expression of an active Src. MKN28 treated with Midostaurin or Bosutinib significantly suppressed migration in vitro and in zebrafish models. Immunohistochemical analyses of PHF8, PKCα, and PTEN showed a positive correlation between PHF8 and PKCα. Moreover, high PHF8-PKCα expression is significantly correlated with worse prognosis. Together, the present invention suggests that the PHF8-PKCα-Src-PTEN pathway is a prognostic/therapeutic target in HER2-negative advanced GC.

Unless otherwise defined herein, scientific and technical terminologies employed in the present invention shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise.

Therefore, the present invention provides a method of inhibiting tumor progression in a subject suffering from gastric cancer, comprising administering to said subject a pharmaceutical composition comprising an inhibitor of targeting PHF8-c-Jun-PKCα-Src-PTEN axis, or a pharmaceutically acceptable salt thereof. In a preferred embodiment, the tumor progression comprises tumor growth, cancer dissemination, and metastasis.

In another embodiment, the gastric cancer is HER2-negative gastric cancer.

In a preferred embodiment, the inhibitor is a PHF8 inhibitor. In a preferred embodiment the PHF8 inhibitor is a nucleotide inhibitor adapted to inhibit PHF8 expression. In a more preferred embodiment, the nucleotide inhibitor is a RNA oligonucleotide inhibitor, comprising a small interfering RNA (siRNA), a short hairpin RNA (shRNA), or a micro RNA oligonucleotide (miRNA).

In another embodiment, the inhibitor is capable disrupting the interaction between PHF8 and c-Jun thereby inhibiting to activate PRKCA expression.

In another embodiment, the inhibitor is a PKCα inhibitor. In a preferred embodiment, the PKCα inhibitor is Midostaurin.

In another preferred embodiment, the inhibitor is a Src inhibitor. In a preferred embodiment, the Src inhibitor is Bosutinib.

In another embodiment, the inhibitor is a combination of PKCα inhibitor and a Src inhibitor. In a preferred embodiment, the inhibitor is a combination of Midostaurin and Bosutinib.

The present invention also provides a method of determining a tumor progression state in a subject suffering from gastric cancer, comprising: (a) providing a sample from the subject; (b) detecting PHF8 expression level in the sample from the subject; and (c) determining the tumor progression state of gastric cancer by the PHF8 expression level, wherein the PHF8 expression level is from moderate to strong expression indicating the subject suffering a late stage of gastric cancer.

In one embodiment, the PHF8 expression level is PHF8 gene or PHF8 protein expression.

In a preferred embodiment, the PHF8 expression level is the PHF8 gene expression level. In a more preferred embodiment, the PHF8 gene expression level is determined by quantitative real-time PCR or in situ hybridization.

In a preferred embodiment, the PHF8 expression level is the PHF8 protein expression. In a more preferred embodiment, the PHF8 protein expression level is determined by immunoblotting, immunohistochemistry, or immunomagnetic reduction. In a more preferred embodiment, the PHF8 protein expression level is determined by immunohistochemistry.

In another embodiment, the determining of the positively detection of the PHF8 is based on immunoreactive score (IRS), which is calculated by multiplying the staining intensity by the proportion of positive cells. In one embodiment, the immunoreactive score (IRS) is score based on the intensity grade (score: 1-3) and the proportion of positive tumor cells (score: 1-4).

In one embodiment, the IRS value is calculated between 1-4 means weak expression, the IRS value between 6-8 means moderate expression, and the IRS value between 9-12 means strong expression.

In one embodiment, the late stage of gastric cancer is from stage II to stage IV. In another embodiment, the late stage of gastric cancer means tumor with lymph node metastasis or distant metastasis.

### EXAMPLES

The examples below are non-limiting and are merely representative of various aspects and features of the present invention.

Materials and Methods

Cell Culture

Human adenocarcinoma cell lines, MKN28 (JCRB no. JCRB0253) and MKN45 (JCRB no. JCRB0254), were cultured in RPMI 1640 medium (Thermo, Waltham, Mass., USA) supplied with 10% fetal bovine serum (Hyclone, Logan Utah, USA) at 37° C. in 5% CO2. 293T (ATCC no. CRL-3216), the human embryonic kidney cell line, was cultured in DMEM medium (Thermo, Waltham, Mass., USA) supplied with 10% fetal bovine serum.

Antibodies, Reagents and Plasmids

Rabbit anti-PHF8 was purchased from B ethyl Laboratories (Montgomery, Tex., USA). Rabbit anti-c-Jun, anti-PTEN, anti-Src and anti-HA were purchased from Cell Signaling Technology (Danvers, Mass., USA). Mouse anti-β-actin and anti-flag were purchased from Sigma-Aldrich (St. Louis, Mo., USA). Rabbit anti-IgG was purchased from Santa Cruz Biotechnology (Dallas, Tex., USA). Rabbit anti-H3K9me2 and anti-H4K20me1 were purchased from Active Motif (Carlsbad, Calif., USA). Rabbit anti-p-Src(Y419) was purchased from Thermo. Rabbit anti-PKCα was purchased from Abcam (Cambridge, Mass., USA). Lentiviral vector pLKO-control (pLKO), pLKO-shPHF8 (shPHF8 #1, TRCN0000118319; shPHF8 #2, TRCN0000118320), shPRKCA (shPRKCA #1, TRCN0000001692; shPRKCA #2, TRCN0000001693) plasmids were purchased from The RNAi Consortium (TRC). Inhibitors Midostaurin and Bosutinib were purchased from Sigma-Aldrich. pHACE-PKCα (Addgene plasmid #21232), pCDNA3-SRC (Addgene plasmid #44652) p6600-c-Jun (Addgene plasmid #34898)

Establishment of Knockdown Cells

Lentivirus particles were produced in 293T cells using pLP1, pLP2, and pLP/VSVG packaging system (Thermo) according to the user's manual. MKN28 and MKN45 cells were infected with lentivirus carrying pLKO, shPHF8, or shPRKCA, and followed by puromycin selection (2 μg/ml). The knockdown efficiency was evaluated by immunoblotting analysis.

Migration Assay and Chemoresponse Assay in Zebrafish

The Tg (fli1: EGFP) zebrafish transgenic strain was purchased from ZIRC (Oregon, USA). The zebrafish embryos, larvae, and adult fish were maintained in the Taiwan Zebrafish Core Facility @ NHRI. At two-day post-fertilization, the embryos were dechorionated and subsequently anesthetized with tricaine (0.04 mg/ml). MKN28 or MKN45 cells were labeled with either CFSE or CM-DiI. Two hundred labeled cells (4.6 nl) were injected into the yolk of two-day old embryo using a Nanoject II Auto-Nanoliter Injector (Drummond Science, Broomall, Pa., USA). Later, individual recipients were examined for fluorescent cells 1 and 3 day-post injection by a fluorescence microscopy (Leica). The animal protocols for zebrafish experiments were approved by the IACUC of the NHRI (IACUC number: IACUC-107057-AC1).

Cell Proliferation Assay and Trans-Well Migration Assay

Cell proliferation was measured by seeding 1×105 cells per well of a six-well plate, followed by counting the cell numbers on day 0, 24, 48, 72, and 96 hr. Migration assays were performed in Transwell 24-well plates with the 8-μm filters (BD, Franklin Lakes, N.J., USA). 7×104 cells (diluted in 200 μl 0.5% medium) were seeded in the upper chamber, and 500 μl of complete medium was added to the lower chamber. After 24-hour incubation, the non-migrating cells on the upside were removed with a cotton swab, and cells on the underside that were examined and detected by crystal violet staining.

Mice Xenograft and Inhibitory Experiment

Five million MKN28 cells (pLKO, shPHF8 #1, or shPHF8 #2) were injected subcutaneously with matrigel at both flanks of five nude mice for each group. Two weeks after implantation, the tumor volume was measured every week for six weeks. The animal protocols for mice experiments were approved by the Institutional Animal Care Use Committee (IACUC) of the NHRI.

Immunoblotting and Immunoprecipitation Assay

For immunoblotting assay, PBS-washed cells collected by scraping were directly lysed in RIPA buffer supplemented with protease inhibitor (Santa Cruz) and PhosSTOP (Sigma). Appropriate amounts of protein were separated on SDS-PAGE and then electrotransferred to PVDF membrane (PALL, Protein Washington, N.Y., USA). After the transfer, each membrane was incubated with appropriate primary antibody overnight at 4° C. and then probed with secondary antibodies conjugated with fluorescence. Odyssey Infrared Imaging System (LI-COR Biosciences, Lincoln, Nebr., USA) was utilized to detect the level of fluorescence. For immunoprecipitation assay, cell pellets were lysed in lysis buffer [50 mM Tris-HCl (pH 7.4), 150 mM NaCl, 0.5% NP40)] containing protease inhibitor (Santa Cruz). The lysates were then incubated with the corresponding antibodies (1 μg) as indicated and 10 μl of PureProteome protein A/G magnetic beads (Millipore) at 4° C. overnight. The beads were washed with IP-wash buffer (137 mM NaCl, 2.7 mM KCL, 10 mM Na2HPO4, 1.8 mM K2HPO4, 0.1% Tween 20, pH 7.4) and eluted by IP-lysis buffer, follow by immunoblotting assay as described above.

ChIP Assay

MKN28 cells were crosslinked with formaldehyde (1%) for 10 min and then quenched in 0.125 M glycine. The cell lysates were fragmented by sonication (100-500 bps), followed by IP analysis using corresponding antibodies as indicated (rabbit IgG, anti PHF8, anti-c-Jun, anti-H3K9me2, and anti-H4K20me1) and Magna ChIP G Magnetic Beads (Millipore) at 4° C. overnight. The precipitated ChIP complexes were analyzed and quantified by using qRT-PCR. The primer sequence and the target site of the indicated gene is showed in Table 1. Fold enrichment was calculated based on threshold cycle (Ct) as 2−Δ(ΔCt), where ΔCt=Ct (IP)−Ct (input) and Δ(ΔCt)=ΔCt (antibody)−ΔCt (IgG).

TABLE 1
Primer sequence and the target site
of the indicated gene for ChIP assay

| SEQ | | | Target |
|---|---|---|---|
| ID No. | Primer name | Sequence (5′ to 3′) | site |
| 1 | AXL_ChIP-F | TAGAGAGACACGGCCTCACTGG | −94 to |
| 2 | AXL_ChIP-R | TCCCAGACTTGGGCAACCCTTT | −309 |
| 3 | HBEGF_ChIP-F | CACCAGTCACTTTCGAAGCGG | +295 to |
| 4 | HBEGF_ChIP-R | CGCCTCCTGGAGCCTTATTCCC | +46 |
| 5 | ICAM-1_ChIP-F | GCTATAAAGGATCACGCGCC | −82 to |
| 6 | ICAM-1_ChIP-R | TGTTGCCTTTCAATCGCTGT | +152 |
| 7 | PRKCA_ChIP-F | AGTCTCTACCACACGCAGGTGA | −94 to |
| 8 | PRKCA-ChIP-R | ACGACGCCCGTGTTTTAGCTG | −925 |
| 9 | ROBO1_ChIP-F | CAATGACAATGGCGTCAGCA | −543 to |
| 10 | ROBO1_ChIP-R | TTGCTAGTCACCACAGGTCG | −748 |
| 11 | SATB2_ChIP-F | AGAACCGGCGTTTCAGATGT | −99 to |
| 12 | SATB2_ChIP-R | CCAGAACACAATCCGGCCT | −308 |
| 13 | TUBE1_ChIP-F | GAAACATGGCCGCTGGATTC | +227 to |
| 14 | TUBE1_ChIP-R | CCTACAGACGTGCTTAGCGA | +45 |
| 15 | UGT8_ChIP-F | TAACGTTTGTGTGCTATGGA | −809 to |
| 16 | UGT8_ChIP-R | ACCGAGAAAGTCTAAGTGTG | −683 |

Microarray

Global expression analysis was performed in MKN28 pLKO vs. shPHF8 #1 (GEO: GSE117980). Microarray analysis was performed by the National Health Research Institutes (NHRI), using Affymertix GeneChip Human Gene 2.0 ST Array (Affymetrix, Santa Clara, Calif., USA). Subsequent analysis was done by DAVID (The Database for Annotation, Visualization and Integrated Discovery) Bioinformatics database and USCS genome browser tool.

Micro-Western Array

Signaling pathway analysis was performed by the Micro-western Core facility of the NHRI, using appropriate antibodies as indicated and followed the protocol described preciously (Ciaccio, Wagner et al., 2010).

mRNA Collection and Quantitative Real-Time PCR (qRT-PCR)

Total RNAs were extracted with TRizol reagent (Thermo). The cDNAs were prepared using the SuperScript III Reverse Transcriptase (Thermo), dNTP (Genedirex, Las Vegas, Nev., USA) and random primers (Thermo). The amount of cDNA samples was detected with SensiMix™ SYBR® Hi-ROX Kit (Bioline, Taunton, Mass., USA) and ABI StepOnePlus Real-Time PCR System (Thermo). GAPDH served as an internal control. The list of primers was provided in Table 2.

TABLE 2
Primer sequence for quantitative real-time PCR

| SEQ ID No. | Primer name | Sequence (5′ to 3′) |
|---|---|---|
| 17 | AXL-F | CGTAACCTCCACCTGGTCTC |
| 18 | AXL-R | TCCCATCGTCTGACAGCA |
| 19 | HBEGF-F | TGGGGCTTCTCATGTTTAGG |
| 20 | HBEGF-R | CATGCCCAACTTCACTTTCTC |
| 21 | ICAM-1-F | CCTTCCTCACCGTGTACTGG |
| 22 | ICAM-1-R | AGCGTAGGGTAAGGTTCTTGC |
| 23 | PTEN-F | GGCTAAGTGAAGATGACAATC |
| 24 | PTEN-R | GTTACTCCCTTTTTGTCTCTG |
| 25 | PRKCA-F | TCGACTGGGAAAAACTGGAG |
| 26 | PRKCA-R | CTCTGCTCCTTTGCCACAC |
| 27 | ROBO1-F | CGATGGAGGAAAGATGATGG |
| 28 | ROBO1-R | CAAGGTATGATCATCTCGGATTT |
| 29 | SATB2-F | GCCCTGGGGTATTCTCACA |
| 30 | SATB2-R | ACTGAGGGGGAGAGGGTTC |
| 31 | TUBE1-F | TTCTTTAGAAATGTGGACACCAGA |
| 32 | TUBE1-R | TTAAAGAACATATTTTTCCCTTGGA |
| 33 | UGT8-F | CTATGAAGCACTAGTGAAGGTTATCAA |
| 34 | UGT8-R | CCTTGTGAATTTCCGAAAGC |

IHC and IHC Scoring

Three consecutive paraffin embedded human GC biopsies (n=42) were obtained from Chang Sung Memorial Hospital (CGMH), Taoyuan, Taiwan. Tissue array ST1505 (n=50) was purchased from US Biomax. The information of the embedded human GC biopsies was shown in Table 3. IHC staining was performed using anti-PHF8, anti-PKCα and anti-PTEN and scored by a qualified pathologist. IHC results were scored based on the intensity grade (score: 1-3) and the proportion of positive tumor cells (score: 1-4). Immunoreactive score (IRS) was then calculated by multiplying the staining intensity by the proportion of positive cells. The present invention was approved by institutional Review Board of Chang Sung Memorial Hospital (IRB number: 201800374B00501).

TABLE 3
The information of the embedded human GC biopsies.

| | CGMH | | Biomax tissue array ST1505 | |
|---|---|---|---|---|
| Characteristics | No. | % | No | % |
| Gender | | | | |
| Female | 18 | 42.9 | 8 | 16.0 |
| Male | 24 | 57.1 | 42 | 84.0 |
| Tumor stage | | | | |
| I | 12 | 28.6 | 10 | 20.0 |
| II | 7 | 16.7 | 35 | 70.0 |
| III | 22 | 52.4 | 4 | 8.0 |
| IV | 1 | 2.4 | 1 | 2.0 |
| T stage | | | | |
| T1 | 6 | 14.3 | 4 | 8.0 |
| T2 | 8 | 19.1 | 9 | 18.0 |
| T3 | 8 | 19.1 | 34 | 68.0 |
| T4 | 20 | 47.6 | 3 | 6.0 |
| N stage | | | | |
| N0 | 14 | 33.3 | 44 | 88.00 |
| N1 | 10 | 23.8 | 6 | 12.0 |
| N2 | 7 | 16.7 | 0 | 0.0 |
| N3 | 11 | 26.2 | 0 | 0.0 |

Statistical Analysis

The student's t-test was used to calculate the statistical significance of the experimental results between two groups (significance at p<0.05). Overall survival and disease-free progression were analyzed by log-rank test. Chi-square test was used to compare groups with categorical variables in IHC analysis. p-value of comparison between tumor and PHF8 expression was calculated by one-way ANOVA.

### Example 1. Overexpression of PHF8 is Associated with Worse Clinical Outcomes in HER2-Negative Gastric Cancer

The present invention first evaluated the expression values of 21 KDMs in normal gastric mucosa and tumor tissues from the selected studies obtained from the Oncomine database (www.oncomine.org/) (Table 4).

TABLE 4
Statistical analysis of expression of KDMs based on Oncomine

| | | | No. of | No. of | |
|---|---|---|---|---|---|
| | | Sample | normal | cancer | |
| Gene symbol | Dataset | type | sample | sample | p value |
| KDM1A | Chen_2003 | Unpaired | 29 | 72 | 0.004 |
| | Derrico_2009 | Unpaired | 31 | 38 | 0.609 |
| | Cui_2011 | Paired | 78 | 78 | 0.009 |
| | Cho_2011 | Unpaired | 19 | 65 | 0.137 |
| KDM1B | Chen_2003 | Unpaired | 28 | 103 | p < 0.001 |
| | Derrico_2009 | Unpaired | 31 | 38 | 0.050 |
| | Cho_2011 | Unpaired | 19 | 65 | p < 0.001 |
| KDM2A | Chen_2003 | Unpaired | 27 | 102 | 0.008 |
| | Derrico_2009 | Unpaired | 31 | 38 | 0.005 |
| | Cui_2011 | Paired | 78 | 78 | p < 0.001 |
| KDM2B | Chen_2003 | Unpaired | 29 | 102 | 0.007 |
| | Derrico_2009 | Unpaired | 31 | 38 | p < 0.001 |
| | Cui_2011 | Paired | 78 | 78 | 0.188 |
| | Cho_2011 | Unpaired | 19 | 65 | 0.885 |
| KDM3A | Chen_2003 | Unpaired | 28 | 96 | 0.020 |
| | Derrico_2009 | Unpaired | 31 | 38 | p < 0.001 |
| | Cui_2011 | Paired | 78 | 78 | 0.253 |
| | Cho_2011 | Unpaired | 19 | 65 | 0.100 |
| KDM3B | Chen_2003 | Unpaired | 26 | 102 | 0.132 |
| | Derrico_2009 | Unpaired | 31 | 38 | p < 0.001 |
| | Cui_2011 | Paired | 78 | 78 | 0.383 |
| | Cho_2011 | Unpaired | 19 | 65 | 0.879 |
| KDM4A | Chen_2003 | Unpaired | 24 | 90 | 0.666 |
| | Derrico_2009 | Unpaired | 31 | 38 | 0.521 |
| | Cui_2011 | Paired | 78 | 78 | 0.110 |
| | Cho_2011 | Unpaired | 19 | 65 | 0.034 |
| KDM4B | Chen_2003 | Unpaired | 17 | 93 | 0.227 |
| | Derrico_2009 | Unpaired | 31 | 38 | 0.636 |
| | Cui_2011 | Paired | 78 | 78 | 0.012 |
| | Cho_2011 | Unpaired | 19 | 65 | 0.085 |
| KDM4C | Chen_2003 | Unpaired | 20 | 92 | 0.511 |
| | Derrico_2009 | Unpaired | 31 | 38 | 0.669 |
| | Cui_2011 | Paired | 78 | 78 | 0.079 |
| | Cho_2011 | Unpaired | 19 | 65 | 0.889 |
| KDM4D | Derrico_2009 | Unpaired | 31 | 38 | 0.169 |
| | Cui_2011 | Paired | 78 | 78 | 0.750 |
| | Cho_2011 | Unpaired | 19 | 65 | 0.032 |
| KDM5A | Chen_2003 | Unpaired | 27 | 102 | 0.900 |
| | Derrico_2009 | Unpaired | 31 | 38 | p < 0.001 |
| | Cui_2011 | Paired | 78 | 78 | 0.062 |
| | Cho_2011 | Unpaired | 19 | 65 | 0.484 |
| KDM5B | Chen_2003 | Unpaired | 29 | 103 | 0.163 |
| | Derrico_2009 | Unpaired | 31 | 38 | 0.169 |
| | Cui_2011 | Paired | 78 | 78 | 0.005 |
| | Cho_2011 | Unpaired | 19 | 65 | 0.149 |
| KDM5C | Derrico_2009 | Unpaired | 31 | 38 | 0.493 |
| | Cui_2011 | Paired | 78 | 78 | 0.005 |
| | Cho_2011 | Unpaired | 19 | 65 | p < 0.001 |
| KDM5D | Chen_2003 | Unpaired | 20 | 85 | 0.061 |
| | Derrico_2009 | Unpaired | 31 | 38 | 0.858 |
| | Cho_2011 | Unpaired | 19 | 65 | 0.035 |
| KDM6A | Derrico_2009 | Unpaired | 31 | 38 | 0.953 |
| | Cui_2011 | Paired | 78 | 78 | 0.836 |
| | Cho_2011 | Unpaired | 19 | 65 | 0.346 |
| KDM6B | Chen_2003 | Unpaired | 27 | 101 | 0.289 |
| | Derrico_2009 | Unpaired | 31 | 38 | 0.005 |
| | Cui_2011 | Paired | 78 | 78 | 0.731 |
| | Cho_2011 | Unpaired | 19 | 65 | 0.129 |
| KDM7A | Derrico_2009 | Unpaired | 31 | 38 | 0.905 |
| KDM7B | Chen_2003 | Unpaired | 28 | 95 | p < 0.001 |
| | Derrico_2009 | Unpaired | 31 | 38 | p < 0.001 |
| | Cui_2011 | Paired | 78 | 78 | p < 0.001 |
| KDM7B | Cho_2011 | Unpaired | 19 | 65 | 0.018 |
| KDM7C | Derrico_2009 | Unpaired | 31 | 38 | 0.043 |
| | Cui_2011 | Paired | 78 | 78 | 0.140 |
| | Cho_2011 | Unpaired | 19 | 65 | 0.636 |
| KDM8 | Chen_2003 | Unpaired | 26 | 102 | 0.076 |
| | Derrico_2009 | Unpaired | 31 | 38 | p < 0.001 |
| | Cui_2011 | Paired | 78 | 78 | 0.383 |
| | Cho_2011 | Unpaired | 19 | 65 | 0.879 |
| KDM9 | Chen_2003 | Unpaired | 19 | 88 | 0.268 |
| | Derrico_2009 | Unpaired | 31 | 38 | p < 0.001 |
| | Cui_2011 | Paired | 78 | 78 | 0.110 |
| | Cho_2011 | Unpaired | 19 | 65 | 0.213 |

The results shown as Table 4 were that KDM1B, KDM2A and PHF8/KDM7B were significantly up-regulated in tumor specimens compared to normal tissues (p<0.05). Next, the clinical relevance was evaluated of the upregulated KDMs with respect to the endpoints of 5-year overall survival (OS) and first progression (FP) for HER2-negative patients with GC using data retrieved from Kaplan-Meier Plotter (KM Plotter) (Table 5).

TABLE 5
Clinical outcomes of selected KDMs in Kaplan-Meier Plotter analysis

| | | Survival | No. of | | 95% | |
|---|---|---|---|---|---|---|
| Gene symbol | Affy ID | outcome | cases | HR | CI | p value |
| KDM1B | 1553150_at | OS | 429 | 1.59 | 1.17-2.16 | 0.025 |
| | | FP | 356 | 1.72 | 1.21-2.45 | p < 0.001 |
| | 227021_at | OS | 429 | 0.70 | 0.52-0.92 | 0.016 |
| | | FP | 356 | 0.64 | 0.46-0.91 | 0.006 |
| KDM2A | 208987_s_at | OS | 532 | 1.80 | 1.43-2.27 | p < 0.001 |
| | | FP | 408 | 1.72 | 1.32-2.24 | p < 0.001 |
| | 208988_at | OS | 532 | 0.75 | 0.59-0.95 | 0.014 |
| | | FP | 408 | 0.68 | 0.50-0.93 | 0.007 |
| | 208989_s_at | OS | 532 | 1.48 | 1.13-1.93 | 0.002 |
| | | FP | 408 | 1.42 | 1.04-1.95 | 0.016 |
| KDM7B | 212916_at | OS | 532 | 1.57 | 1.21-2.04 | p < 0.001 |
| | | FP | 408 | 1.54 | 1.71-2.02 | 0.002 |
| | 215065_at | OS | 532 | 1.92 | 1.47-2.51 | p < 0.001 |
| | | FP | 408 | 2.08 | 1.47-2.95 | p < 0.001 |

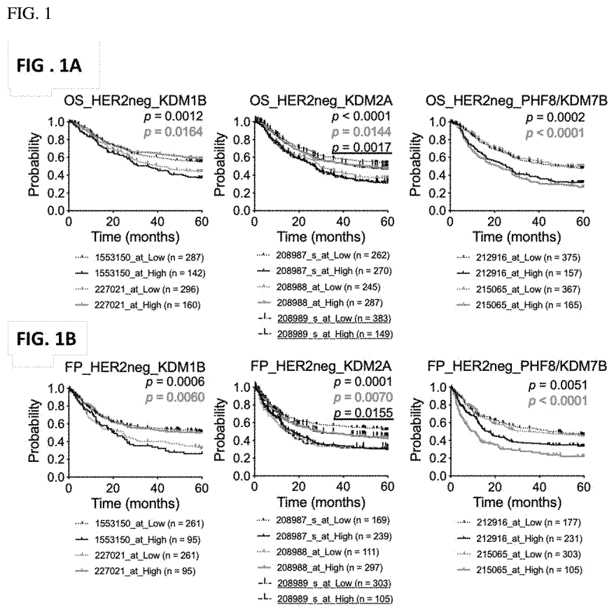

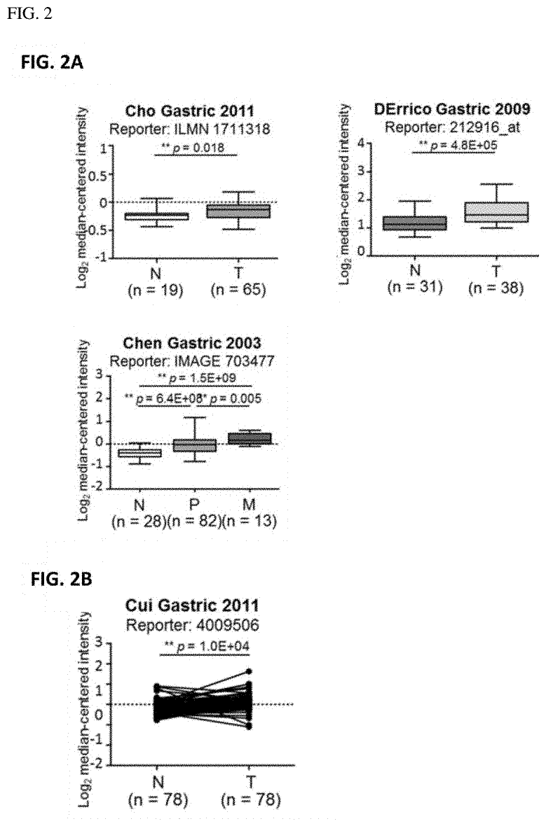

Higher PHF8/KDM7B expression was significantly associated with worse OS for HER2-negative cases. KDM1B and KDM2A; however, exhibited inconsistent results depending on the probe (FIGS. 1A and 1B). Furthermore, an even significantly higher level of PHF8/KDM7B was noted in sites of metastasis than in primary-tumor sites (FIG. 2A). Additionally, PHF8/KDM7B expression was higher in tumor tissues than in adjacent normal tissues (FIG. 2B). These results together indicate that PHF8 was a prognostic epigenetic regulator in Her2-negative GC.

### Example 2. Effects of PHF8 in Metastatic GC Cells In Vitro and In Vivo

PHF8 was noted to have an even significantly higher expression at the metastatic sites of GC in Oncomine analysis. To characterize the biological role of PHF8 in GC progression, two HER2-negative, metastatic MKN28 and MKN45 lines resembling the chromosomally unstable tumors (CIN) subtype were utilized. MKN45 was obtained from the liver metastasis of a patient with a poorly differentiated primary GC of diffuse histology and was characterized as microsatellite unstable tumors (MSI)-low and Epstein-Barr Virus (EBV)-negative. MKN28 was derived from a lymph node metastasis with an intestinal differentiation primary GC and had moderate copy number alterations and a moderate number of genes with single nucleotide variants.

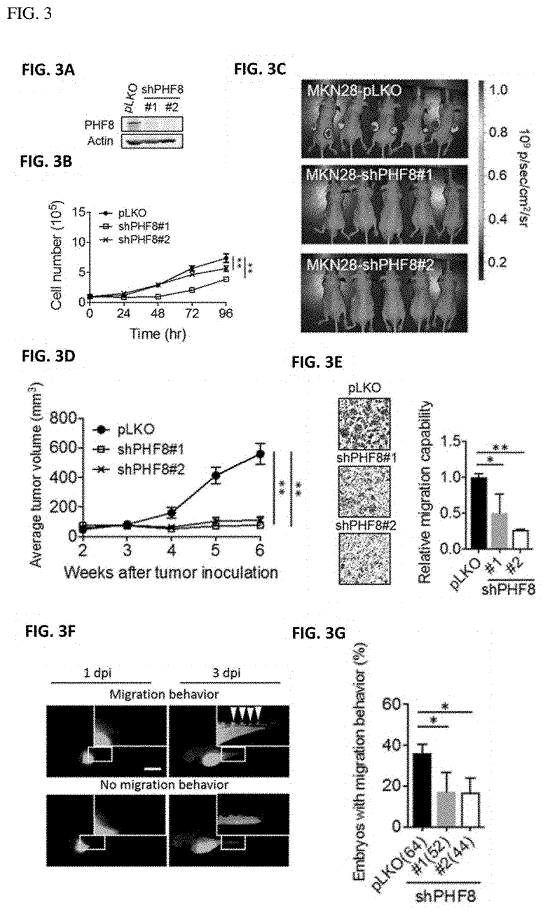

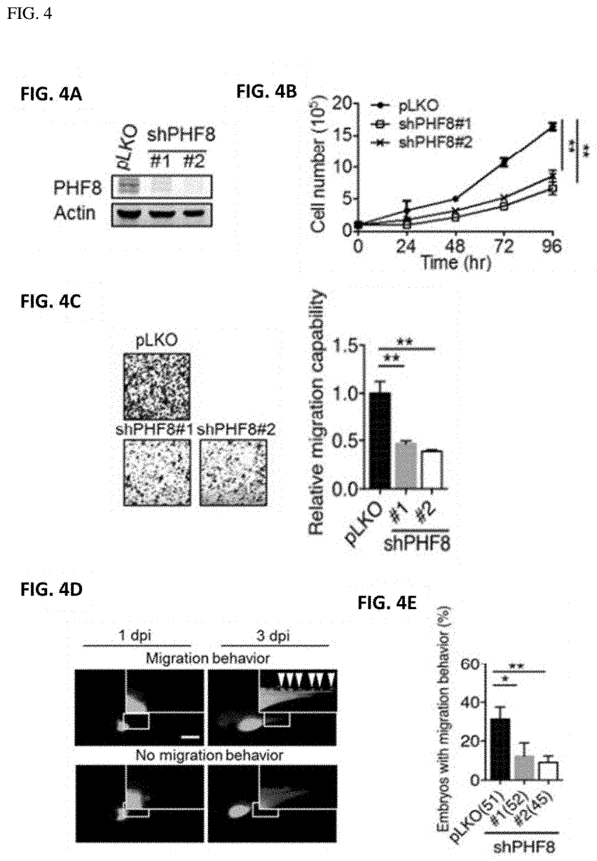

Control (pLKO) or PHF8-depleted cells (shPHF8 #1 and shPHF8 #2) were created for MKN28 by using a lentiviral approach (FIG. 3A). The depletion of PHF8 significantly reduced the levels of cell proliferation (FIG. 3B) and those of migration (FIG. 3E) as compared with those of control cells. Importantly, tumor growth of MKN28 xenografts was significantly impaired for the PHF8-KD MKN28 cells (FIG. 3D). Similar results also found for MKN45 PHF8-depleted (shPHF8 #1 and shPHF8 #2) cells as compared with those of control cells (FIGS. 4A-4C).

Next, to evaluate migration behavior using a zebrafish xenotransplantation assay, an efficient in vivo system that utilizes a small number of cells (100 to 200 cells) to accurately monitor cell migratory activity within a couple of days. Cells (pLKO vs. shPHF8) were labeled with carboxyfluorescein succinimidyl ester (CFSE), an amine-reactive green fluorescent dye, and injected into zebrafish embryos. Migration activity was monitored at 1-day postinjection (1 dpi) and 3 dpi by fluorescence microscopy. As shown in FIGS. 3F and 4D, control cells disseminated to the distal parts at 3 dpi (upper panel) as compared with cells that remained in the embryo. The quantification of embryos harboring distal tumor foci revealed a significantly higher degree of metastatic activity for the pLKO group (36.1%) than for the shPFH8 group (shPHF8 #1, 17.1%; shPHF8 #2, 16.7%) (FIG. 3G). Likewise, there was a significantly lower degree of migration in the PHF8-knockdown MKN45 group than in the control group (pLKO, 31.3%; shPHF8 #1, 8.9%; and shPHF8 #2, 11.8%) (FIG. 4E). The present invention found that PHF8 is crucial for tumor growth and migration.

### Example 3

PHF8 Promotes GC Progression by Regulating PKCα and ICAM-1

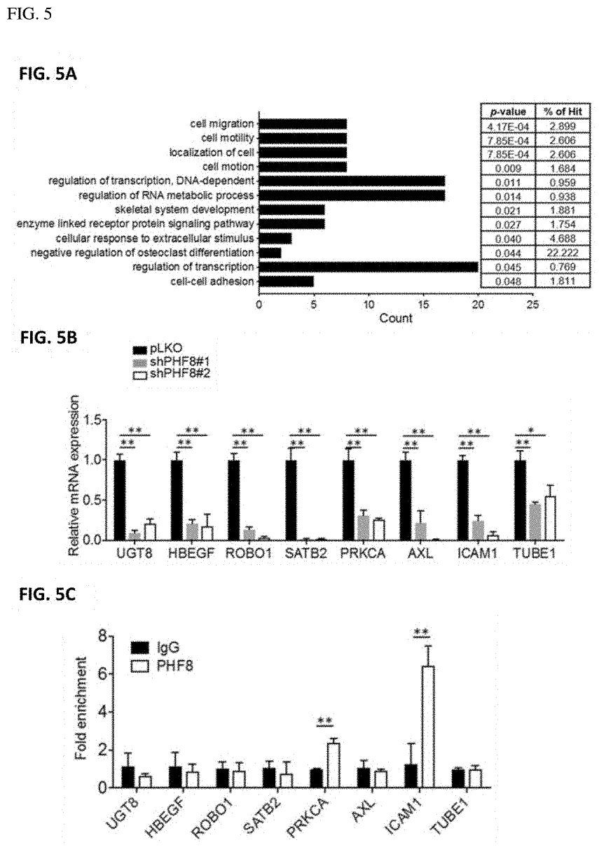

To characterize the molecular mechanisms by which PHF8 contributes to GC progression, a comparative microarray analysis of pLKO and shPHF8 MKN28 cells (GSE117980) were performed. DAVID functional annotation indicated that genes that were down-regulated (less than or equal to twofold; n=150) in shPHF8 cells were primarily involved in cell migration (n=8, P=0.00041) and cell motility (n=8, P=0.00079) (https://david.ncifcrf.gov/) (FIG. 5A). qRT-PCR confirmed that the eight genes associated with both cell migration and motility (UGT8, HBEGF, ROBO1, STATB2, PRKCA, AXL, ICAM1, and TUBE1) had significantly lower expression levels in two independent shPHF8 cell lines than in pLKO cells (FIG. 5B).

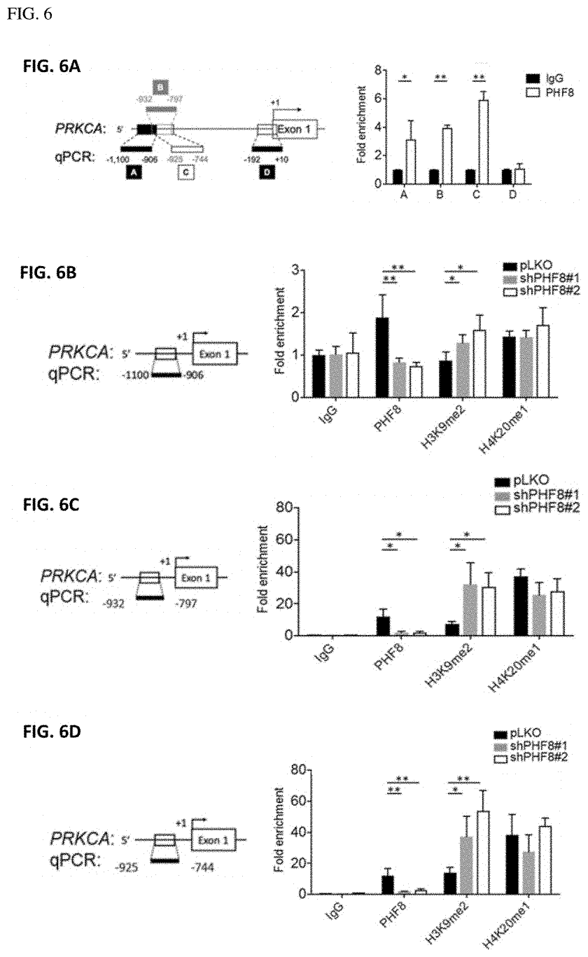

Next, to evaluate whether PHF8 was directly involved in regulating the expression of the genes identified in the microarray analysis. In a chromatin immunoprecipitation (ChIP) analysis, there was a significantly higher signal of PHF8 than of IgG on the promoter regions of PRKCA and ICAM-1 in MKN28 (FIG. 5C). An additional ChIP analysis with four sets of primers designed across the promoter region of PRKCA in MKN28 revealed positive PHF8-binding signals between −1,100-bp and −744-bp regions, particularly at the site of −925 to −744 bp (FIG. 6A). The ChIP signals of H3K9me2 and H4K20me1 on the PRKCA locus in cells without or with depletion of PHF8 were compared. As shown in FIGS. 6B-6D, the H3K9me2 signal but not the H4K20me1 was significantly increased in two independent shPHF8 lines, suggesting that PHF8 regulated the expression of PRKCA by erasing the repressive H3K9me2 mark on the PRKCA locus.

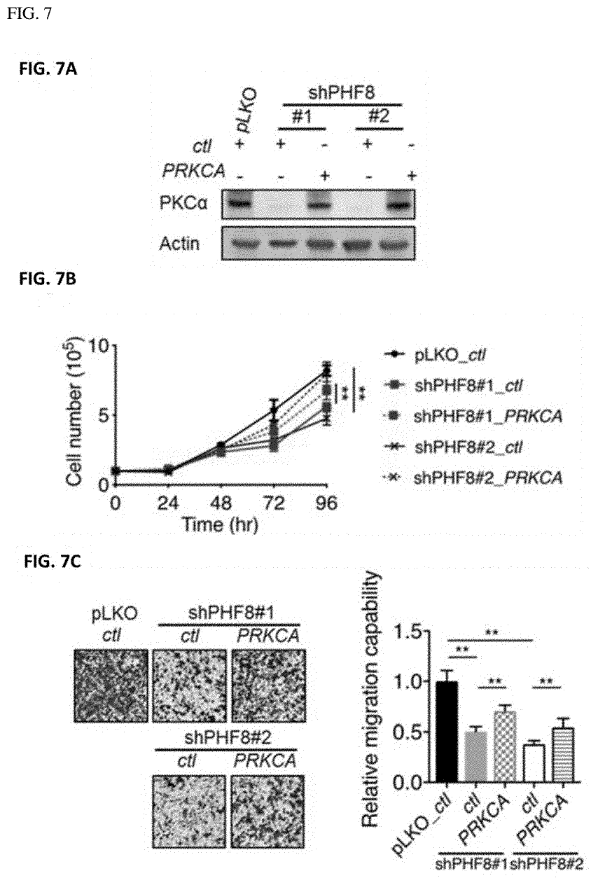

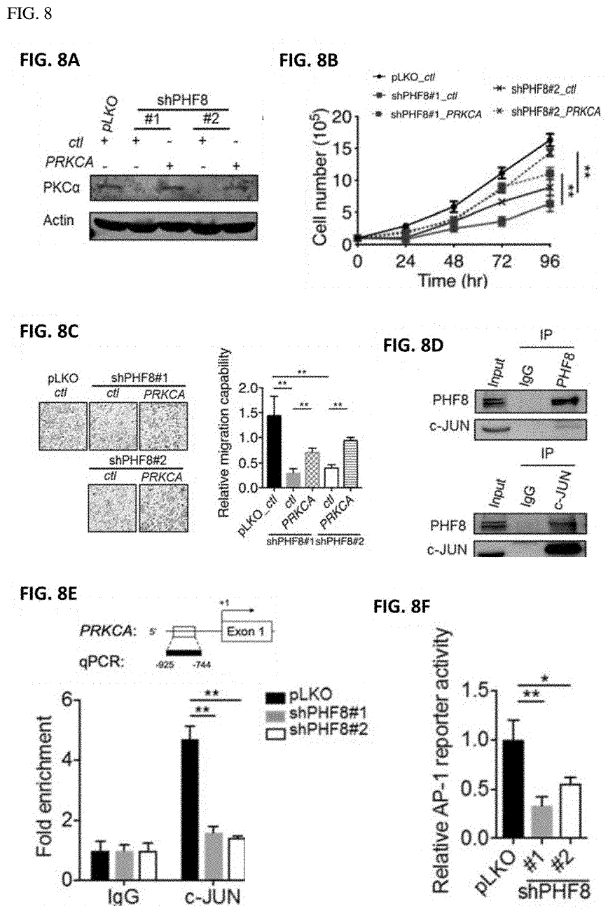

To substantiate that PRKCA acted as a downstream target of PHF8, it ectopically expressed PKCα in each of the two shPHF8 lines (MKN28, FIG. 7A and MKN45, FIG. 8A). Interestingly, the ectopic expression of PKCα in shPHF8 significantly restored the levels of cell proliferation (MKN28, FIG. 7B and MKN45, FIG. 8B) and migration (MKN28, FIG. 7C and MKN45, FIG. 8C) as compared to those of the pLKO line. Collectively, the present invention suggested that PHF8 displayed H3K9me2 demethylation activity to up-regulate the expression of PRKCA involved in cell proliferation and migration.

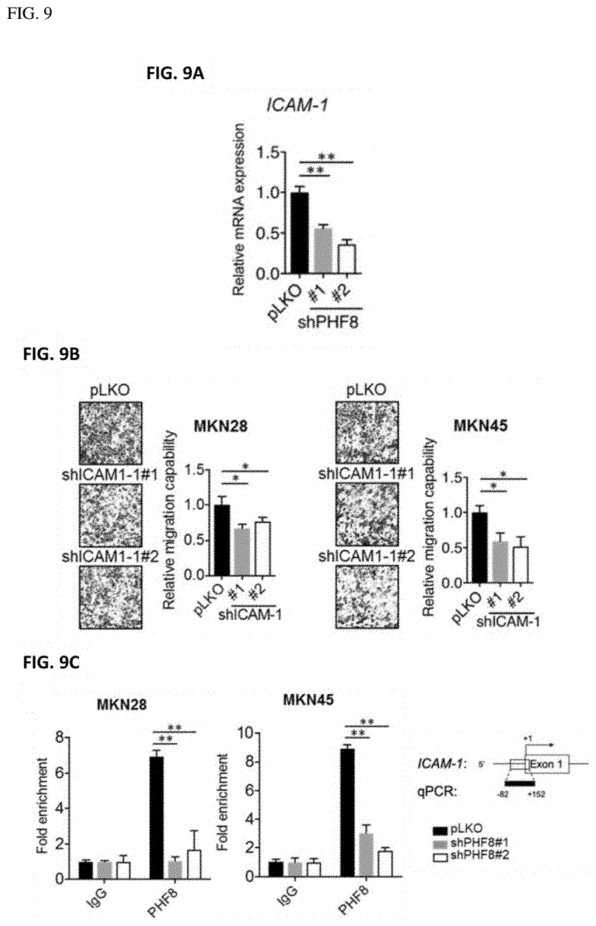

In addition, to evaluate whether PHF8 directly regulated the expression of ICAM-1. FIG. 9A showed that the depletion of PHF8 reduced the expression of ICAM-1 mRNA in MKN45, consistent with that observed in MKN28 (FIG. 5B). Moreover, PHF8's signal in the promoter region of ICAM-1 using a ChIP analysis had been detected (FIG. 9B). The depletion of ICAM-1 led to a decreased degree of migratory effect as compared to the control (FIG. 9C), indicating that PHF8-regulated expression of ICAM-1 also contributed to GC progression.

### Example 4

PHF8 Interacts with c-Jun and they are Co-Recruited to the PRKCA Locus

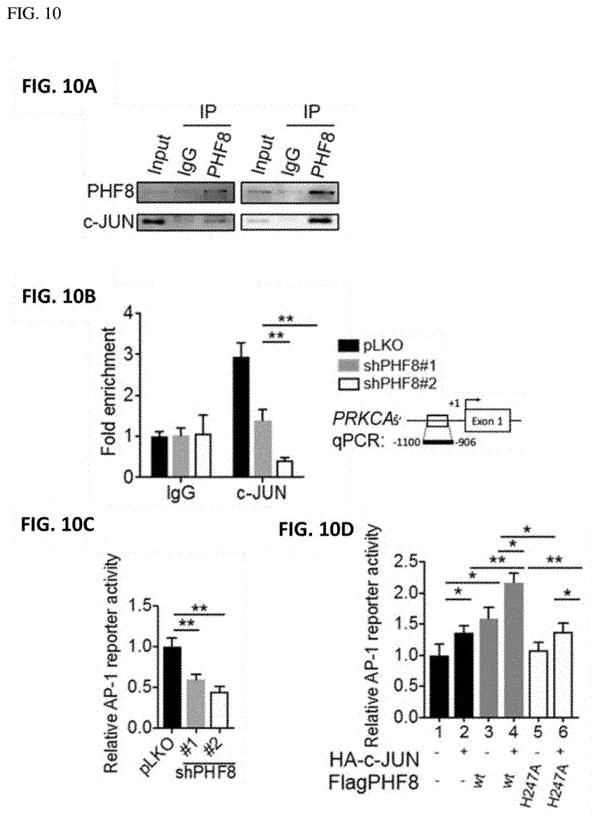

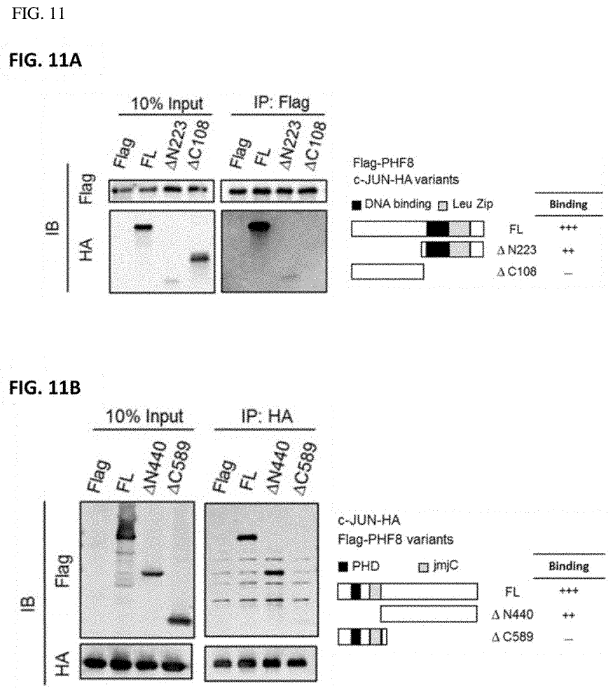

Next to identify potential transcription factors interacting with PHF8 by using the University of California Santa Cruz Genome Browser. c-Jun, a component of the AP-1 transcription factor, showed positive binding peaks in the promoter region of PRKCA in two ChIP-Seq datasets (ChIP-Seq from A549 (ENCLB202COI) (Ab: PHF8): GSM2700325; ChIP-Seq from A549 (ENCLB403GIO) (Ab: c-Jun): GSM2437720). Immunoprecipitation (IP) of lysates from MKN28 or MKN45 using either anti-PHF8 or anti-c-Jun antibodies indeed revealed that endogenous PHF8 was associated with c-Jun (MKN28, FIG. 10A and MKN45, FIG. 8D). Further to determine the crucial region of PHF8 that interacted with c-Jun. The full-length PHF8 and various truncated variants fused with a Flag tag (Flag-tagged full-length [FL], N-terminal [Δ N440], and C-terminal [ΔC589] truncated variants) were generated. IP analysis revealed that the C-terminal region of PHF8 (residues 441 to 1,024) was most critical for its interaction with c-Jun (FIG. 11A). The reciprocal experiment for c-Jun by generating full-length and truncated c-Jun variants fused to an HA-tag (HA-tag FL, N-terminal [ΔN223], and C-terminal [ΔC108] truncated forms) were performed. IP analysis showed that c-Jun Δ N223, but not ΔC108, retained the association with PHF8, indicating that the C-terminal region of c-Jun (residues 224 to 331) was critical for the PHF8-c-Jun interaction (FIG. 11B).

To support the notion that PHF8 acted as a coactivator of c-Jun and regulated the expression of PRKCA, a ChIP analysis was conducted for pLKO and shPHF8 cells using anti-c-Jun and IgG for comparison. Statistically significant enrichment of c-Jun was detected in pLKO cells at the PRKCA locus (MKN28, FIG. 10B and MKN45, FIG. 8E). Of note, a c-Jun/AP-1 binding site was found in the promoter region of PRKCA based on the ChIP-Seq data (Gene Expression Omnibus [GEO] accession no. GSM2437720). Using the AP-1 reporter activity assay, the depletion of PHF8 significantly reduced the trans-activation of AP-1 reporter activity (MKN28, FIG. 10C and MKN45, FIG. 8F). Next to examine whether overexpressing PHF8 and/or c-Jun stimulates AP-1 transcriptional activity. FIG. 10D showed that overexpressing PHF8 or c-Jun alone significantly enhanced the level of AP-1 transactivation. Remarkably, there was an even significantly pronounced increase when overexpressing PHF8 with c-Jun. Interestingly, this enhancement was not seen when overexpressing an inactive mutant, PHF8(H247A) (FIG. 10D). The present invention found that PHF8 modulates the expression of PRKCA in conjunction with c-Jun through PHF8's demethylase activity.

### Example 5

The PHF8-PKCα Axis Regulates PTEN Destabilization Via Src

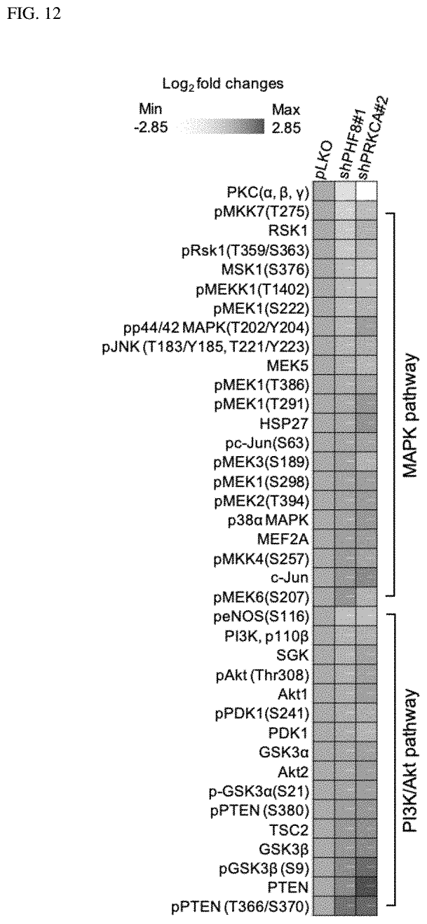

PRKCA encodes PKCα, a serine/threonine protein kinase that serves as a signaling molecule activated by Ca²⁺ and phospholipids; accordingly, to explore the signaling pathway mediated by the PHF8-PKCα axis. A Western microarray analysis was used to evaluate the patterns of 96 antibodies simultaneously in pLKO, shPHF8, and shPRKCA cells. FIG. 12 showed that two pathways were altered substantially: PI3K and MAPK. In particular, the tumor suppressor PTEN/pPTEN had the highest signal intensities in both shPHF8 and shPRKCA cells.

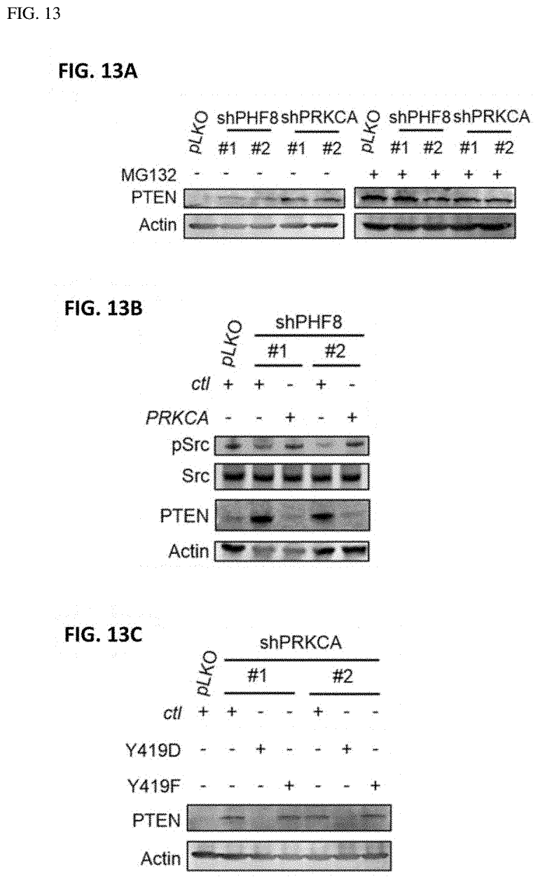

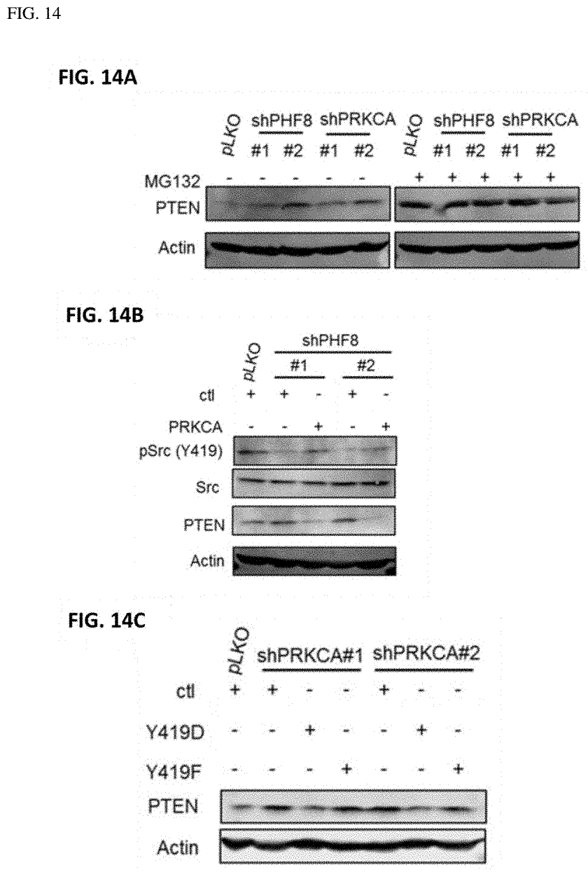

Western blotting analyses of pLKO and shPHF8 lines confirmed that PTEN was clearly up-regulated by the depletion of PHF8 (MKN28, FIG. 13A and MKN45, FIG. 14A). Moreover, the level of PTEN was substantially higher in each of the two shPRKCA lines than in pLKO, indicating that PHF8-PKCα signaling led to PTEN destabilization (MKN28, FIG. 13A and MKN45, FIG. 14A). Therefore, to ask whether PTEN was regulated at the level of transcriptional silencing or translational modification in the context of the PHF8-PKCα axis. First to evaluate whether treatment with MG132, a proteasome inhibitor could restore the PTEN signal, since PTEN was quite susceptible to proteasomal degradation, particularly upon posttranslational modifications. The protein level of PTEN was indeed rescued in MG132-treated pLKO cells (MKN28, FIG. 13A and MKN45, FIG. 14A). Analyses of the abundance of Src and activated Src (pY419) in pLKO and two shPHF8 lines, interestingly, revealed that there was indeed a reduced level of activated Src (pY419) in PHF8-depleted cells (MKN28, FIG. 13B and MKN45, FIG. 14B). Furthermore, complementation with PKC α using a PRKα-expressing vector in each of the two shPHF8 lines restored the level of activated Src (MKN28, FIG. 13B and MKN45, FIG. 14B), supporting the notion that Src served as a downstream effector of PKCα. The introduction of a constitutively active Src variant (Y419D), but not a kinase-dead Src variant (Y419F) greatly diminished the abundance of PTEN (MKN28, FIG. 13C and MKN45, FIG. 14C). Together, the present invention found that PHF8 negatively regulates PTEN destabilization via the PKCα-Src-induced signaling pathway.

### Example 6

Targeting the PKCα-Src Pathway in Metastatic GC.

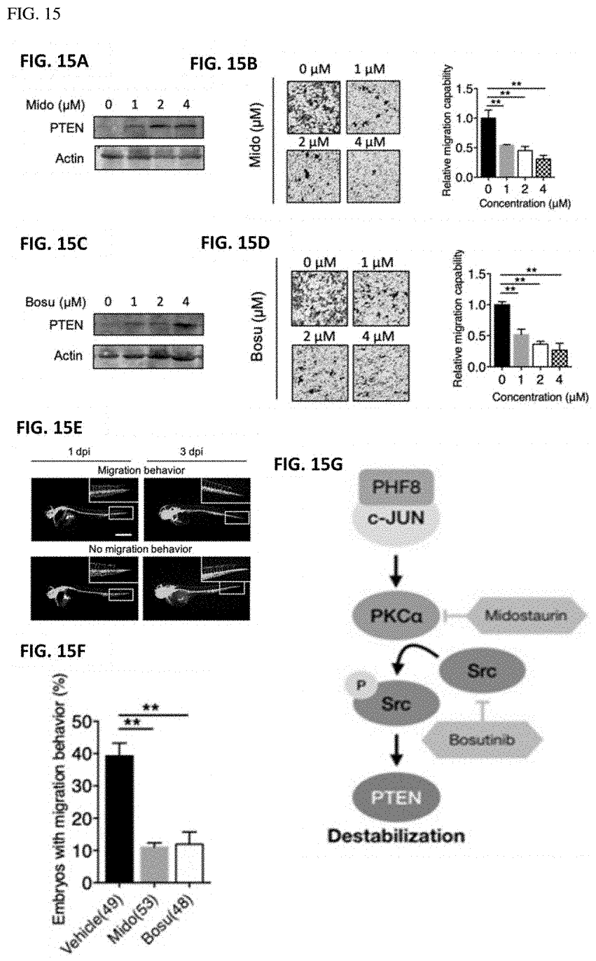

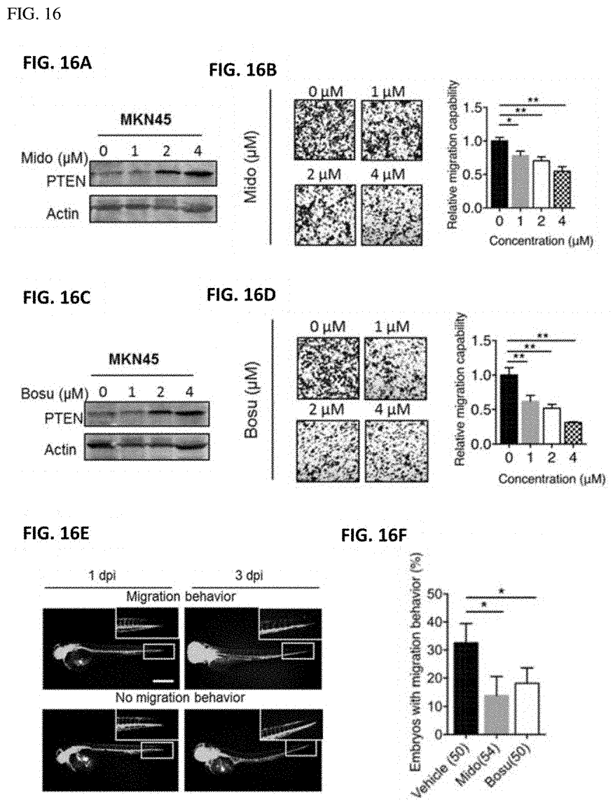

To test whether targeting the PHF8-PKCα-Src-PTEN axis using pharmacological inhibitors curbs GC metastasis. Treatment with Midostaurin, a PKC a inhibitor indeed led to an elevated level of PTEN in a dose-dependent manner in MKN28 (FIG. 15A) and in MKN45 (FIG. 16A). Importantly, the degree of cell migration was also significantly suppressed (MKN28, FIG. 15B and MKN45, FIG. 16B). Likewise, the inhibition of Src by Bosutinib resulted in an increased level of PTEN expression in both MKN28 (FIG. 15C) and MKN45 (FIG. 16C). As such, there was also a reduced degree of cell migration (MKN28, FIG. 15D and MKN45, FIG. 16D).

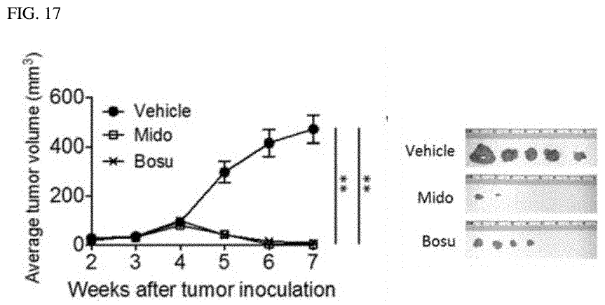

Next, to corroborate this finding using a zebrafish xeno-transplantation model. Cells labeled with Vybrant CM-DiI (CM-Dil) (red fluorescence dye) were injected into the embryos of Tg (fli1: EGFP) (fish with green fluorescence in blood vessels), followed by immersion in solutions containing 1 μM m Midostaurin or Bosutinib (a sublethal dose) at 1 dpi. Implantation of MKN28 or MKN45 cells in embryos often resulted in cell dissemination; cells clearly metastasized to distal parts of the body (MKN28, FIG. 15E and MKN45, FIG. 16E). A large proportion of MKN28-injected embryos treated by Midostaurin or Bosutinib had reduced levels of widespread dissemination and invasion (39.3% in the vehicle group, 11% in the Midostaurin group, 11.95% in the Bosutinib group) (FIG. 15F). MKN45 injection also resulted in a significantly higher levels of metastatic activity in the vehicle group (34.25%) than in the two inhibitor groups (13.88% in the Midostaurin group and 18.08% in the Bosutinib group) (FIG. 16F). Next, to test whether Midostaurin or Bosutinib impedes tumor growth in vivo by using a MKN28 xenograft model. FIG. 17 showed that both drugs significantly impaired tumor growth as compared to vehicle group. Collectively, the present invention suggested that the inhibition of PKC a or Src was an effective strategy to curb tumor progression in vivo.

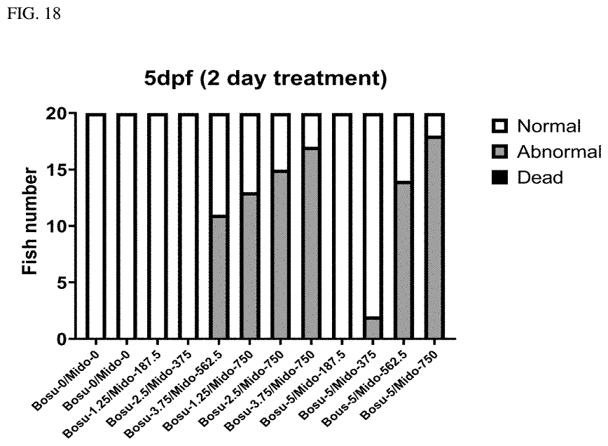

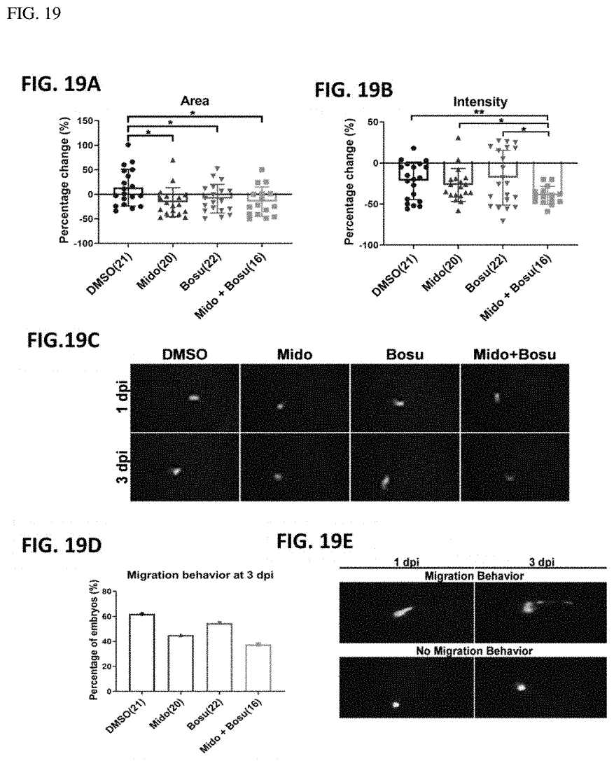

To test the sublethal dosage of combing Midostaurin and Bosutinib for targeting the PKCα-Src pathway in vivo model of zebrafish embryos. The present invention showed that Midostaurin at 187.5 nM combined with Bosutinib at 1.25 μM had no toxicity for zebrafish embryo treated for 2 days starting 3 dpf. Therefore, the dosage was chosen for xenotransplantation assay (FIG. 18). In addition, the results showed Midostaurin had synergetic effect with Bosutinib for MKN28 to inhibit the cell proliferation and migration in vivo (FIG. 19). The Midostaurin at 187.5 nM reduced cell proliferation of MKN28, Bosutinib at 1250 nM had little effect on cell proliferation; however, Midostaurin (187.5 nM) with Bosutinib (1250 nM) exhibited a statistical significantly reduced cell proliferation of MKN28 in zebrafish embryos (FIGS. 19A-19C). The Midostaurin at 187.5 nM exhibited a reduced proportion of embryos with migration behavior in MKN28, Bosutinib at 1250 nM had little effect on cell migration, Midostaurin (187.5 nM) with Bosutinib (1250 nM) exhibited a significantly reduced proportion of embryos with migration behavior in MKN28 (FIGS. 19D-19E).

### Example 7

Immunohistochemically Analyses of PHF8, PKCα, and PTEN in GC Subjects.

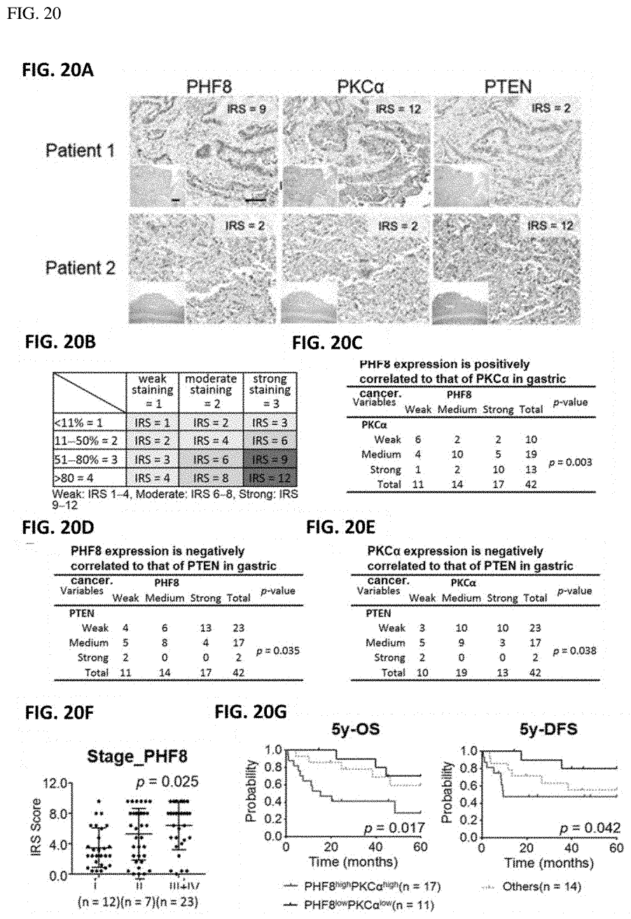

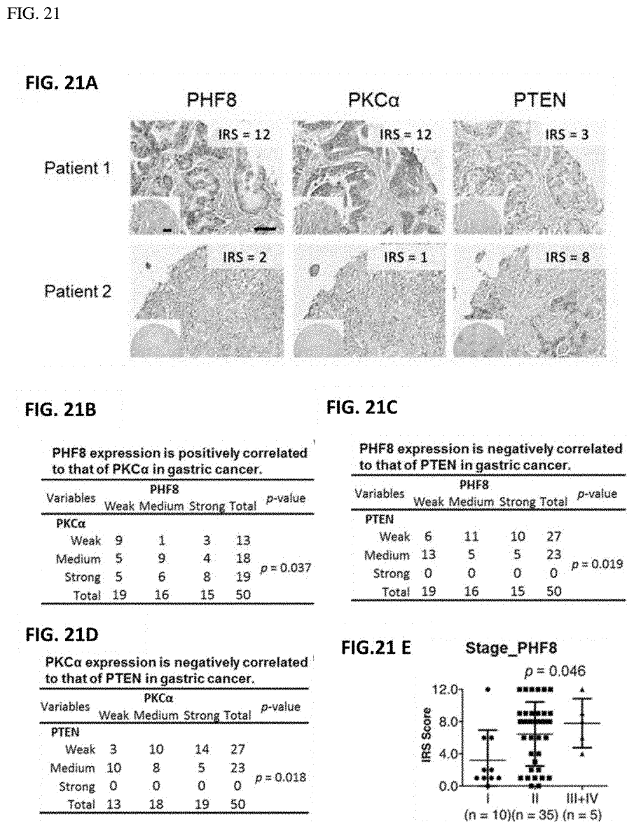

Given the role of the PHF8-PKCα-PTEN axis in the progression of HER2-negative GC in vitro and in vivo, the clinical relevance of these markers were evaluated by using IHC analysis of PHF8, PKCα, and PTEN for a large sample of patient with GC (subjects obtained from CGMH, FIG. 20 and Biomax tissue array ST1505, FIG. 21). IHC results were scored based on two parameters: the intensity grade (score: 1-3) and the proportion of positive tumor cells (score: 1-4). The immunoreactive score (IRS) was obtained by multiplying the intensity grade by the positive proportion score (FIGS. 20A-20B and FIG. 21A). Remarkably, there were significant correlations between these markers (FIGS. 20C-20E and FIGS. 21B-21D), i.e., a positive correlation between PHF8 and PKCα and negative correlations between PHF8 and PTEN and between PKCα and PTEN. Of note, PHF8 abundance was significantly positively correlated with tumor stage (FIG. 20F and FIG. 21E). PHF8 significantly predicted a poor OS and disease-free survival. Furthermore, the PHF8^{high}PKCα^{high} group (IRS score ≥6) was significantly associated with a poor OS and disease-free survival (FIG. 20G).

What is claimed is:

1. A method of inhibiting tumor progression in a subject suffering from gastric cancer, comprising administering to said subject a pharmaceutical composition comprising an inhibitor of targeting PHF8-c-Jun-PKCα-Src-PTEN axis, or a pharmaceutically acceptable salt thereof.

2. The method of claim 1, wherein the tumor progression comprises tumor growth, cancer dissemination, and metastasis.

3. The method of claim 1, wherein the gastric cancer is HER2-negative gastric cancer.

4. The method of claim 1, wherein the inhibitor is capable disrupting the interaction between PHF8 and c-Jun thereby inhibiting to activate PRKCA expression.

5. The method of claim 1, wherein the inhibitor is a PKCα inhibitor.

6. The method of claim 1, wherein the inhibitor is a Src inhibitor.

7. The method of claim 1, wherein the inhibitor is a combination of PKCα inhibitor and a Src inhibitor.

8. The method of claim 5, wherein the PKCα inhibitor is Midostaurin.

9. The method of claim 6, wherein the Src inhibitor is Bosutinib.

10. A method of determining a tumor progression state in a subject suffering from gastric cancer, comprising:
(a) providing a sample from the subject;
(b) detecting PHF8 expression level in the sample from the subject; and
(c) determining the tumor progression state of gastric cancer by the PHF8 expression level, wherein the PHF8 expression level is positively detected from moderate to strong expression indicating the subject suffering a late stage of gastric cancer.

11. The method of claim 10, wherein the PHF8 expression level is PHF8 gene expression or PHF8 protein expression.

12. The method of claim 11, wherein the PHF8 gene expression is determined by quantitative real-time PCR or in situ hybridization.

13. The method of claim 11, wherein the PHF8 protein expression is determined by immunoblotting, immunohistochemistry, or immunomagnetic reduction.

14. The method of claim 10, wherein further comprises detecting PKCα expression level, wherein the PKCα expression level are from moderate to strong expression predicting poor prognosis in the subject suffering from gastric cancer.

15. The method of claim 10, wherein the late stage of gastric cancer is from stage II to stage IV.

16. The method of claim 10, wherein the late stage of gastric cancer is tumor with lymph node metastasis or distant metastasis.


SEQUENCE LISTING (34 sequences)

SEQ ID NO: 1 - DNA, 22 bp, Artificial Sequence
  AXL F primer for ChIP
tagagagacacggcctcactgg

SEQ ID NO: 2 - DNA, 22 bp, Artificial Sequence
  AXL R primer for ChIP
tcccagacttgggcaacccttt

SEQ ID NO: 3 - DNA, 21 bp, Artificial Sequence
  HBEGFF primer for ChIP
caccagtcactttcgaagcgg

SEQ ID NO: 4 - DNA, 22 bp, Artificial Sequence
  HBEGF R primer for ChIP
cgcctcctggagccttattccc

SEQ ID NO: 5 - DNA, 20 bp, Artificial Sequence
  ICAM-1 F primer for ChIP
gctataaaggatcacgcgcc

SEQ ID NO: 6 - DNA, 20 bp, Artificial Sequence
  ICAM-1 R primer for ChIP
tgttgcctttcaatcgctgt

SEQ ID NO: 7 - DNA, 22 bp, Artificial Sequence
  PRKCA F primer for ChIP
agtctctaccacacgcaggtga

SEQ ID NO: 8 - DNA, 21 bp, Artificial Sequence
  PRKCA R primer for ChIP
acgacgcccgtgttttagctg

SEQ ID NO: 9 - DNA, 20 bp, Artificial Sequence
  ROBO1 F primer for ChIP
caatgacaatggcgtcagca

SEQ ID NO: 10 - DNA, 20 bp, Artificial Sequence
  RROBO1 primer for ChIP
ttgctagtcaccacaggtcg

SEQ ID NO: 11 - DNA, 20 bp, Artificial Sequence
  SATB2 F primer for ChIP
agaaccggcgtttcagatgt

SEQ ID NO: 12 - DNA, 19 bp, Artificial Sequence
  SATB2 R primer for ChIP
ccagaacacaatccggcct

SEQ ID NO: 13 - DNA, 20 bp, Artificial Sequence
  TUBE1 F primer for ChIP
gaaacatggccgctggattc

SEQ ID NO: 14 - DNA, 20 bp, Artificial Sequence
  TUBE1 R primer for ChIP
cctacagacgtgcttagcga

SEQ ID NO: 15 - DNA, 20 bp, Artificial Sequence
  UGT8 F primer for ChIP
taacgtttgtgtgctatgga

SEQ ID NO: 16 - DNA, 20 bp, Artificial Sequence
  UGT8 R primer for ChIP
accgagaaagtctaagtgtg

SEQ ID NO: 17 - DNA, 20 bp, Artificial Sequence
  AXL F primer for PCR
cgtaacctccacctggtctc

SEQ ID NO: 18 - DNA, 18 bp, Artificial Sequence
  AXL R primer for PCR
tcccatcgtctgacagca

SEQ ID NO: 19 - DNA, 20 bp, Artificial Sequence
  HBEGF F primer for PCR
tggggcttctcatgtttagg

SEQ ID NO: 20 - DNA, 21 bp, Artificial Sequence
  HBEGF R primer for PCR
catgcccaacttcactttctc

SEQ ID NO: 21 - DNA, 20 bp, Artificial Sequence
  ICAM-1 F primer for PCR
ccttcctcaccgtgtactgg

SEQ ID NO: 22 - DNA, 21 bp, Artificial Sequence
  ICAM-1 R primer for PCR
agcgtagggtaaggttcttgc

SEQ ID NO: 23 - DNA, 21 bp, Artificial Sequence
  PTEN  F primer for PCR
ggctaagtgaagatgacaatc

SEQ ID NO: 24 - DNA, 21 bp, Artificial Sequence
  PTEN R primer for PCR
gttactccctttttgtctctg

SEQ ID NO: 25 - DNA, 20 bp, Artificial Sequence
  PRKCA F primer for PCR
tcgactgggaaaaactggag

SEQ ID NO: 26 - DNA, 19 bp, Artificial Sequence
  PRKCA R primer for PCR
ctctgctcctttgccacac

SEQ ID NO: 27 - DNA, 20 bp, Artificial Sequence
  ROBO1 F primer for PCR
cgatggaggaaagatgatgg

SEQ ID NO: 28 - DNA, 23 bp, Artificial Sequence
  ROBO1 R primer for PCR
caaggtatgatcatctcggattt

SEQ ID NO: 29 - DNA, 19 bp, Artificial Sequence
  SATB2 F primer for PCR
gccctggggtattctcaca

SEQ ID NO: 30 - DNA, 19 bp, Artificial Sequence
  SATB2 R primer for PCR
actgagggggagagggttc

SEQ ID NO: 31 - DNA, 24 bp, Artificial Sequence
  TUBE1 F primer for PCR
ttctttagaaatgtggacaccaga

SEQ ID NO: 32 - DNA, 25 bp, Artificial Sequence
  TUBE1 R primer for PCR
ttaaagaacatatttttcccttgga

SEQ ID NO: 33 - DNA, 27 bp, Artificial Sequence
  UGT8 F primer for PCR
ctatgaagcactagtgaaggttatcaa

SEQ ID NO: 34 - DNA, 20 bp, Artificial Sequence
  UGT8 R primer for PCR
ccttgtgaatttccgaaagc